(19) United States
(12) Patent Application Publication
(10) Pub. No.: US 2011/0050228 A1
(43) Pub. Date: Mar. 3, 2011

(54) AGENT FOR TRANSPORTING NUCLEAR SPIN ORDER AND FOR MAGNETIC RESONANCE IMAGING

(75) Inventors: Malcolm Harris Levitt, Southampton (GB); Giuseppe Pileio, Southampton (GB)

(73) Assignee: University of Souhampton, Southhmpton (GB)

(21) Appl. No.: 12/921,880

(22) PCT Filed: Mar. 10, 2009
(86) PCT No.: PCT/GB09/00644

§ 371 Date: Nov. 17, 2010

(30) Foreign Application Priority Data
Mar. 10, 2008 (GB) 0804422.4

(51) Int. Cl. G01R 33/44; A61K 49/08; C12Q 1/02; C01B 21/22

(52) U.S. Cl. 324/310; 424/9.33; 435/29; 423/400

(57) ABSTRACT

An agent for magnetic resonance studies, the agent comprising hyperpolarized ¹⁵N labelled N₂O in solution or liquid ¹⁵N—N₂O.

The present invention is concerned with the field of nuclear magnetic resonance (NMR), including magnetic resonance imaging (MRI). More specifically, the present invention concerns an agent that provides a new way of storing and transporting nuclear spin order in the liquid state, and for generating magnetic resonance images with improved characteristics for imaging blood flow and blood oxygenation levels.

Nuclear magnetic resonance relies on the existence of a population difference between the nuclear spin energy levels in a large applied magnetic field. In conventional NMR and NMR imaging, this population difference is generated by establishing thermal equilibrium between the nuclear spins and the local environment. These thermal population differences are very small in accessible magnetic fields, since the relevant energy splitting is much smaller than ordinary thermal energies. The population difference is established on the timescale of a time constant known as the spin-lattice relaxation time and conventionally denoted T₁. The value of T₁ depends on the nuclear spin species and the molecular environment. It is typically in the range of a few tens of milliseconds to several minutes.

In NMR and MRI experiments, the nuclear spin system is perturbed by applying radiofrequency fields. This process generates NMR signals that are proportional to the starting population differences. Since thermal equilibrium only generates a small population difference, the ordinary NMR and MRI signals are correspondingly weak.

Several techniques have been developed that generate much larger nuclear spin population differences than are achievable by the thermal equilibrium route. These include (a) optical pumping of noble gas atoms, (b) dynamic nuclear polarization (DNP) and Overhauser effect methods, which involve microwave irradiation of a cold substance (at a few degrees K) that has been doped with a paramagnetic species, (c) chemical reactions of parahydrogen-enriched hydrogen gas, (d) cooling of a sample to extremely low temperatures (milliKelvin or less) in the presence of paramagnetic dopants, followed by rapid warming. All of these methods have been demonstrated to generate nuclear spin population differences that are many orders of magnitude larger than the ordinary thermally-polarized population differences, with correspondingly larger NMR signals as a result.

Hyperpolarized nuclear spin magnetization returns to the much weaker thermally-polarized magnetization in a time of the order of T₁. The NMR or MRI experiment must therefore be within a time of the order of T₁ or less after the hyperpolarization is established, in order to take advantage of the hyperpolarization enhancement. It will be appreciated by those skilled in the art that a time of the order of T₁ encompasses 2 T₁ or 3 T₁. MRI experiments are now conducted in which a ¹³C-labelled metabolite such as ¹³C-pyruvate is hyperpolarized using the DNP procedure and rapidly injected into an animal or human subject in solution form. Since the ¹³C T₁ of pyruvate is of the order of 30 seconds in solution, one has less than 2 minutes to warm the sample to ambient temperature, transfer the sample from the DNP polarizer and introduce it into the blood stream of the subject, allow it to propagate within the body, and conduct the MRI experiment. This procedure has proved to be feasible and provides informative images.

In systems of two or more coupled spins, the spin-lattice relaxation of the system is more complicated and can in general no longer be characterized by a single time constant T₁. M. Carravetta, O. G. Johannessen and M. H. Levitt, Phys. Rev. Lett. 92, 153003 (2004); M. Carravetta and M. H. Levitt, J. Am. Chem. Soc. 126, 6228-6229 (2004); M. Carravetta and M. H. Levitt, J. Chem. Phys. 122, 214505 (2005) showed that in systems of two coupled spins-½, a nuclear singlet state may be generated that is antisymmetric with respect to exchange of the two nuclear spins, and that in some cases, this singlet state relaxes with a time constant T_{S} that may be more than order of magnitude longer than T₁. In order to reveal these long lifetimes, experiments were designed in which the singlet state is protected from spontaneous conversion into the symmetric nuclear triplet state, which is much shorter-lived. Two such processes were described in the papers cited above, namely (a) transport of the sample into a low magnetic field, and (b) application of a resonant radiofrequency field. Providing these precautions are taken, the singlet state provides a mechanism for preserving hyperpolarized spin order for times much longer than the conventional relaxation time T₁. Such extended lifetimes will make it much easier to administer a hyperpolarized agent in an NMR experiment, and provide much more time for dispersal in the object of interest and for generating an NMR image.

However, there are strong conditions on the type of molecule that supports a long-lived singlet state. The molecular system must contain two magnetic spins-½, in inequivalent molecular sites, and no other nuclei displaying strong nuclear magnetism. Since such systems are not readily available, this phenomenon has so far not found much application.

A molecular system is described herein that (i) provides a long conventional T₁ in solution of between 20 seconds and several minutes, depending on the solvent, (ii) provides a long singlet lifetime T_{S} of around 15 minutes or more, in diamagnetic solution, and (iii) is non-toxic and suitable for administration to human subjects.

In a first aspect, the present invention provides an agent for magnetic resonance studies, the agent comprising hyperpolarized ¹⁵N—N₂O in solution or liquid ¹⁵N—N₂O.

The most common isotope of nitrogen is ¹⁴N, which has a nuclear spin quantum number of 1 and which is unsuitable for most NMR and MRI studies. However, the rare nitrogen isotope ¹⁵N has spin-½ and is suitable for NMR studies, and it is technically feasible to synthesize ¹⁵N-labelled nitrous oxide, ¹⁵N—N₂O.

In ¹⁵N-labelled nitrous oxide, ¹⁵N—N₂O, the two nitrogen sites are inequivalent. There are three ¹⁵N-labelled isotopomers of nitrous oxide, namely [1-¹⁵N]—N₂O, [2-¹⁵N]—N₂O, and [1,2-¹⁵N₂]—N₂O. The singly-labelled isotopomers [1-¹⁵N]—N₂O and [2-¹⁵N]—N₂O display long conventional ¹⁵N T₁'s of more than 20 seconds, and are suitable for conventional hyperpolarization experiments of the type already performed on materials such as ¹³C-pyruvate, as described above. The doubly-labelled isotopomer [1,2-¹⁵N₂]—N₂O also has a long conventional ¹⁵N T₁ of more than 20 seconds, but also supports a long-lived singlet state which has been observed to have a lifetime of more than 26 minutes in low magnetic field (around 3 milliTesla), in a typical diamagnetic solution (deuterated dimethyl sulfoxide).

N₂O is well known as “laughing gas” and is widely used as an anaesthetic and food additive. It is non-toxic and non-addictive, and approved for human use. It is soluble up to a concentration of around 20 milliMolar in water, blood, fat and oil. The chemical replacement of the abundant ¹⁴N isotope with ¹⁵N has no significant chemical or physiological effects. The physiological effects of any of the hyperpolarized ¹⁵N—N₂O isotopomers are indistinguishable from those of non-labelled nitrous oxide.

It is possible to perform NMR experiments on hyperpolarized ¹⁵N—N₂O providing that the NMR or MRI instrument is capable of exciting and detecting signals at the ¹⁵N resonance frequency, which is approximately ten times lower than the conventional proton resonance frequency, measured at the same magnetic field. Some instruments may require modification or adaptation in order to detect ¹⁵N NMR signals.

Thus, in a second aspect, the present invention provides an NMR apparatus comprising:
means to apply a magnetic field to a sample;
means to apply RF radiation to the sample; and
means to detect an NMR signal arising from the sample due to the application of the magnetic field and RF radiation, wherein the means to apply a magnetic field and the means to apply RF radiation are configured to excite an NMR signal from ¹⁵N—N₂O and the means to detect an NMR signal are configured to detect an NMR signal from ¹⁵N—N₂O.

The signal from singly-labelled ¹⁵N—N₂O, i.e. [1-¹⁵N]—N₂O and [2-¹⁵N]—N₂O will experience a chemical shift dependent on its environment. Thus, the apparatus is preferably further configured to indicate the presence of a chemical shift and comprise means for identifying the environment of said ¹⁵N—N₂O from said measured chemical shift.

In doubly-labelled ¹⁵N—N₂O both ¹⁵N sites will experience a different chemical shift. Further, the dipole-dipole coupling between the two sites or “J-coupling” will cause the ¹⁵N peak to split. This may give rise to various spectral and image distortions depending on the pulse sequence used. In some cases, the two chemically distinct sites of ¹⁵N₂—N₂O will give rise to an apparent doubling of the NMR image. Thus, in this case, an MRI pulse sequence can be designed to either combine the data from the two sites into a single image or to produce two images, one image for each site. For example, the system may produce two different images one for each site or combine the data to give a single image.

In an embodiment, a pulse sequence is used which selects the NMR signal from just one site.

If hyperpolarized magnetization is used (as opposed to a hyperpolarized singlet state), the NMR imaging procedures for any of the three ¹⁵N—N₂O isotopomers are essentially identical to those already in use for hyperpolarized ¹³C-pyruvate. A substance containing ¹⁵N—N₂O and a paramagnetic dopant may be hyperpolarized by cooling the mixture to a few degrees Kelvin in a strong magnetic field, and irradiating with microwaves close to the electron Larmor frequency of the dopant. When the hyperpolarized ¹⁵N magnetization is established, the sample is rapidly warmed and separated from the paramagnetic dopant, for example by pressure-forcing through a semipermeable membrane or by chromatographic separation. If this is done in a time short compared to T₁, much of the hyperpolarized magnetization will remain. The purified hyperpolarized ¹⁵N—N₂O solution may be administered into the subject, for example by injection. Some time may be left for dispersion in the subject before a suitable imaging experiment is performed, for example the echo-planar method which can form NMR images in less than one second.

Hyperpolarisation may be performed by other methods, for example by dissolving ¹⁵N₂O in a hyperpolarized medium, and cooling to form a solid in which polarization transfer can take place between the hyperpolarized solvent and the solute. Hyperpolarized liquid xenon-129, which may be generated by a known optical pumping method is an example of a possible medium.

Standard imaging procedures are available that take advantage of contrast generated by the variation of the relaxation time T₁ in bodily tissues. In particular, the proton relaxation time T₁ tends to be shorter in blood with low oxygen levels since oxy-haemoglobin is less paramagnetic than deoxy-haemoglobin. The ¹⁵N relaxation time T₁ of the ¹⁵N—N₂O isotopomers is more sensitive to the oxygenation level of the blood in which it is dissolved than the usual proton relaxation time. Hyperpolarized ¹⁵N—N₂O imaging will therefore generate images with high contrast for blood oxygenation levels, which will be of advantage in clinical diagnosis and functional MRI studies.

In the case of the [1,2-¹⁵N₂]—N₂O isotopomer, it is also possible to use the hyperpolarized singlet state, which potentially has further advantages due to its significantly longer lifetime T_{S}. Exploitation of the singlet state requires some modifications to the procedures given above.

First, the singlet state must be generated in a hyperpolarized form. This may be done either by (1) generating hyperpolarized magnetization, followed by conversion into the singlet state, or (2) direct generation of the hyperpolarized singlet state.

The conversion of magnetization into singlet order has been described in Phys. Rev. Lett. 92, 153003 (2004) and M. Carravetta and M. H. Levitt, J. Chem. Phys. 122, 214505 (2005). The procedure involves (1) selective inversion of the populations of one of the two coupled sites in high magnetic field, followed by (2) transport of the sample into low magnetic field. The selective inversion step may be performed in several ways, well known to NMR practitioners. One common method involves a weak 180-degree pulse, applied at a frequency resonant with one of the two chemically distinct sites. Another method involves the application of two strong 90-degree pulses, with a delay in between. The second method is described in detail in the articles described above. Those skilled in the art will have no difficulty in devising alternative procedures.

Direct generation of hyperpolarized singlet order occurs without intervention if the degree of polarization is high enough. For example, if a two-spin system is 100% polarized, one-half of the resulting order is in the form of (negative) singlet order. However the amount of singlet order is proportional to the square of the polarization fraction. If the polarization is only 10% efficient, the amount of singlet order is only of the order of 1%. Direct hyperpolarization of singlet order is therefore more difficult to achieve than hyperpolarization of magnetization.

Once the hyperpolarized singlet order is generated, it must be maintained by keeping the sample in a sufficiently weak magnetic field. In the case of [1,2-¹⁵N₂]—N₂O, this magnetic field must be less than around 20 milliTesla. Some magnetic shielding may be necessary to ensure this, in the context of an NMR or MRI facility. Providing the magnetic field is kept low enough, the hyperpolarized singlet state will be stable for a time of the order of T_{S}, which has been measured to be more than 26 minutes in a diamagnetic solvent.

The hyperpolarized singlet state may also be maintained if an oscillating magnetic field with a frequency matching the Larmor precession of the nuclei is applied, as described in M. Carravetta and M. H. Levitt, J. Am. Chem. Soc. 126, 6228-6229 (2004). However, this technique is not favoured for producing singlet state ¹⁵N₂O due to the large ¹⁵N chemical shift difference.

The solution of singlet-hyperpolarized [1,2-¹⁵N₂]—N₂O could be administered to a patient via the bloodstream. In order to maintain the singlet state, this must be done in a low-magnetic field region. The long lifetime of the singlet state will allow a longer time for the hyperpolarized agent to be distributed within the body, compared to the conventionally-hyperpolarized material. This will have advantages for the study of organs which are remote from the site of administration.

When the singlet-hyperpolarized [1,2-¹⁵N₂]—N₂O is moved into the magnetic field (either within the subject, or before administration to the subject), the singlet order is converted naturally into hyperpolarized magnetization, but with opposite signs for the two ¹⁵N sites. A selective 180-degree pulse, of the same type as used for the generation of the singlet state, must therefore be applied before a fast NMR imaging sequence is used to construct the NMR image.

The singlet relaxation time T_{S} is expected to be even more sensitive to the blood oxygenation level than the magnetization relaxation time T₁. Those skilled in the art may readily construct NMR imaging methods that provide image contrast through the value of T_{S} rather than the value of T₁.

Liquid hyperpolarized ¹⁵N—N₂O may also be used. However ¹⁵N—N₂O is only liquid at a pressure of 60 bar (at room temperature), so it is not practical for direct use as a contrast agent for human and animal investigation.

Relaxation times for high pressure ¹⁵N—N₂O including the liquid state are expected to be much longer than those for the low pressure. Thus, it is possible to separate ¹⁵N—N₂O from other agents, such as radicals, etc., by going through the gas phase, providing that the pressure is kept high enough.

Hyperpolarized ¹⁵N—N₂O may also be used indirectly in solution, gaseous or liquid form. In this case, the hyperpolarized ¹⁵N—N₂O is used as a means for conveying and transporting hyperpolarized nuclear spin order, rather than as an agent itself. For example, any of the hyperpolarized ¹⁵N—N₂O isotopomers could be generated in liquid form using suitable conditions of temperature and pressure (at ambient temperature, N₂O liquefies at around 60 bar). Other molecular species, for example containing ¹⁵N, could be dissolved in the liquid hyperpolarized N₂O, and the solution frozen. Known cross-relaxation processes will cause the ¹⁵N hyperpolarization to be transferred from the hyperpolarized ¹⁵N—N₂O to the ¹⁵N-labelled nuclei of the solute. The N₂O could then be evaporated leaving a hyperpolarized solid solute. This could be used in turn for NMR experiments or for hyperpolarized NMR imaging experiments. This procedure may be a more convenient method than direct hyperpolarization of the substance of interest, since it will be easier to rapidly separate the paramagnetic agents from nitrous oxide than from most other chemical substances, since nitrous oxide is a very small linear molecule.

Thus, in a further aspect, the present invention provides a method of hyperpolarizing a substance, the method comprising:
liquefying hyperpolarized ¹⁵N—N₂O,
dissolving the substance to be hyperpolarized in said liquefied ¹⁵N—N₂O; and
removing said ¹⁵N—N₂O.

The ¹⁵N—N₂O may be removed by evaporation.

The present invention will now be described with reference to the following non-limiting embodiments in which:

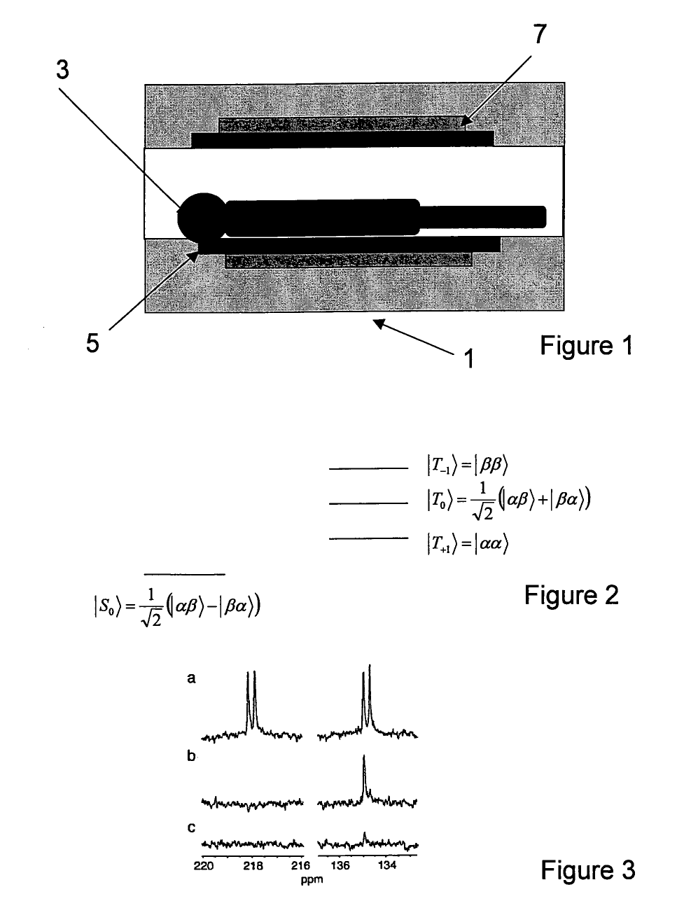

FIG. 1 is a schematic of an MRI apparatus examining a sample in accordance with an embodiment of the present invention;

FIG. 2 is a schematic energy level diagram of ¹⁵N₂—N₂O in low magnetic field, showing the singlet and triplet states;

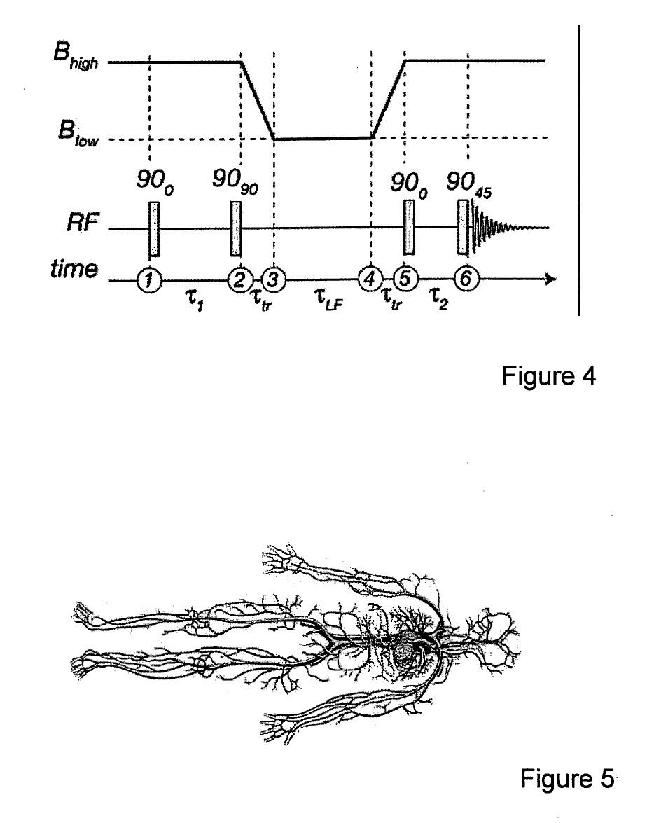

FIG. 3 shows ¹⁵N NMR spectra from ¹⁵N₂—N₂O dissolved in DMSO-d₆, where trace (a) shows the spectrum taken in the convention manner in a magnetic field of 7.04 T and traces (b) and (c) are taken following the procedure summarised in FIG. 4 where τ_{LF}=300 s in trace (b) and τ_{LF}=40 mins in trace (c);

FIG. 4 is a schematic of a pulse sequence used in accordance with an embodiment of the present invention; and

FIG. 5 is a schematic of the circulatory system of a human.

FIG. 1 is a schematic of an MRI apparatus 1 with a human subject 3. The MRI apparatus 1 may be any type of standard MRI apparatus, which comprises a large static magnetic field generally of the order between 1 Tesla and 4.5 Tesla. The static magnetic field is provided by a large magnet. The sample 3 is inserted into the bore of the magnet.

The MRI apparatus 1 comprises a plurality of gradient coils 5. The gradient coils are configured to apply a magnetic gradient across the sample 3 such that different areas of the sample experience a different applied magnetic field. Applying a different field to different parts of the subject allows an image to be produced since the NMR signal produced by the sample 3 is dependent in part on the magnetic field applied to the sample.

The apparatus further comprises RF coils 7 which are configured to subject the sample 3 to RF radiation to excite an NMR response. RF coils are also used to detect the NMR signal. In some MRI apparatus, separate coils are provided to emit and detect RF radiation. In other apparatus, the same coils are used for both functions.

Nuclear magnetic resonance relies on the existence of a population difference between the nuclear spin energy levels in a large applied magnetic field. In NMR and MRI experiments, the nuclear spin system is perturbed by applying radiofrequency fields. This process generates NMR signals that are proportional to the starting population differences.

An agent is used which comprises ¹⁵N-labelled nitrous oxide, ¹⁵N—N₂O. The two nitrogen sites are inequivalent in nitrous oxide. The most common isotope of nitrogen is ¹⁴N, N which has a nuclear spin quantum number of 1 and which is unsuitable for most NMR and MRI studies. There are three ¹⁵N-labelled isotopomers of nitrous oxide, namely [1-¹⁵N]—N₂O, [2-¹⁵N]—N₂O, and [1,2-¹⁵N₂]—N₂O.

For use as an agent a substance containing ¹⁵N—N₂O and a paramagnetic dopant may be hyperpolarized by cooling the mixture to a few degrees Kelvin in a strong magnetic field, and irradiating with microwaves close to the electron Larmor frequency of the dopant. When the hyperpolarized ¹⁵N magnetization is established, the sample is rapidly warmed and separated from the paramagnetic dopant, for example by pressure-forcing through a semipermeable membrane, by chromatographic separation, or by evaporation of the hyperpolarized nitrous oxide. If this is done in a time short compared to T₁, much of the hyperpolarized magnetization will remain.

Other methods of hyperpolarization may be used. For example, hyperpolarized ¹³C-labelled carbon dioxide has been produced by dissolving the ¹³C-labelled carbon dioxide in hyperpolarized liquid xenon-129, which may be generated by a known optical pumping method. The hyperpolarized solution may be solidified by lowering the temperature, and known NMR methodologies may then be used to transfer the polarization between the two nuclear spin species (see C. R. Bowers, H. W. Long, T. Pietrass, H. C. Gaede and A. Pines, “Cross polarization from laser-polarized solid xenon to ¹³CO₂ by low-field thermal mixing,” Chem. Phys. Lett. 205, 168-170 (1993)). The same method should be applicable to ¹⁵N-labelled nitrous oxide.

To maintain the hyperpolarisation before administering to the sample, the hyperpolarized agent is preferably kept under a magnetic field which is higher than the random magnetic fluctuations of the environment where the agent is prepared and transported through. Typically, the agent will be hyperpolarized in the vicinity of the MRI apparatus 1 and the fringe fields of the MRI apparatus 1 satisfy the magnetic field requirements when the agent is being transported and administered to the sample.

The agent is then administered to the sample 3. This may be achieved by a variety of well known techniques. The agent may be directly injected into the sample in gaseous form, or it may be first mixed with blood all and other physiological agent in administered to the sample. Some time may be left for dispersion in the subject before a suitable imaging experiment is performed. However, the method and apparatus may be used to study inanimate objects where other methods of introducing the agent to the sample are used.

Hyperpolarized nuclear spin magnetization returns to the much weaker thermally-polarized magnetization in a time of the order of T₁. The NMR or MRI experiment must therefore be within a time of the order of T₁ or less after the hyperpolarization is established, in order to take advantage of the hyperpolarization enhancement. In this time, if a human or animal sample is the subject of the investigation one must warm the sample to ambient temperature, transfer the sample from a DNP polarizer and introduce it into the blood stream of the subject, allow it to propagate within the body, and conduct the MRI experiment with a T₁ of 3 to 4 minutes in solution, this procedure is feasible.

In order for an NMR signal to be detected from ¹⁵N₂O, the MRI apparatus must be configured to apply an RF field at a frequency which can excite ¹⁵N. The frequency used to excite an NMR response from ¹⁵N is approximately 10 times lower than that used for proton imaging (¹H) using the same magnetic field strength. This is advantageous as lower frequency radio waves penetrate a body more easily. However, lower frequency radiation produces a weaker NMR signal, all other factors being equal. Therefore, in practice, the highest frequency possible will be used which will therefore involve increasing the magnetic field over the level used for proton imaging. For example, magnetic fields of strengths in excess of 4 T may be used, even in excess of 8 or 10 T.

If hyperpolarized magnetization is used (as opposed to a hyperpolarized singlet state), the NMR imaging procedures for any of the three ¹⁵N—N₂O isotopomers are essentially identical to those already in use for hyperpolarized ¹³C-pyruvate. Any of the so-called echo-planar methods which can form NMR images in less than one second may be used.

However, other imaging and spectroscopic analysis methods are possible using ¹⁵N—N₂O. The signal from singly-labelled ¹⁵N—N₂O, i.e. [1-¹⁵N]—N₂O and [2-¹⁵N]—N₂O will experience a chemical shift dependent on its environment. For example, the chemical shift measured when ¹⁵N—N₂O is in fat will be different to that when in blood. This opens up possibilities to analyse the chemical surroundings of ¹⁵N—N₂O from an MRI image.

In doubly-labelled ¹⁵N—N₂O both ¹⁵N sites will experience a different chemical shift. This may give rise to spectral or image distortions depending on the pulse sequence used. Thus, in such cases, an MRI pulse sequence can be designed to either combine the data from the two sites into a single image or to produce two images, one image for each site.

Further, there are techniques available which allow for the study of just one site.

[1,2-¹⁵N₂]—N₂O isotopomer exhibits a singlet state. The singlet state is anti-symmetric under permutation of the two spins of ¹⁵N. In addition to or an alternative to the above, the agent may comprise the hyperpolarized singlet state, which potentially has further advantages due to its significantly longer lifetime T_{S}.

The following lifetimes have been measured for N₂O in solution:

| | T₁(min) at 7.04 T | | |
|---|---|---|---|
| | (times given for | T₁ (min at | Tₛ (min) at |
| N2O in: | each N site | 2 mT) | 2 mT |
| DMSO-d₆ | 1.9/3.1 | 3.3 | 26.4 |
| Olive Oil | 0.7/1.1 | 1.2 | 16.9 |
| Oleic acid | 0.9/1.5 | 1.4 | 16.7 |
| Linoleic acid | 0.9/1.9 | 1.3 | 17.4 |
| Goose fat | 0.4/1.0 | 0.9 | 9.9 |
| Milk cream | 0.9/1.5 | 0.33 | 12.6 |

To generate the singlet state in a hyperpolarized form, first, the hyperpolarized state of ¹⁵N—N₂O may be generated as described above then conversion into the singlet state is performed. This may be achieved by selective inversion of the populations of one of the two coupled sites in high magnetic field, followed by transport of the sample into low magnetic field. The selective inversion step may be performed in several ways, well known to NMR practitioners. One common method involves a weak 180-degree pulse, applied at a frequency resonant with one of the two chemically distinct sites. Another method involves the application of two strong 90-degree pulses, with a delay in between. The weak 180 degree pulse is weak enough to avoid affecting the other of the two N sites.

FIG. 2 is a schematic of the energy level structure of ¹⁵N-labelled nitrous oxide in a low magnetic field. The long-lived nuclear singlet state is shown on the left. The three components of the triplet state are shown on the right. The singlet state is separated from the central triplet state by an energy difference corresponding to the ¹⁵N-¹⁵N J-coupling, which is of the order of 8 Hz. The three triplet states are separated by the ¹⁵N Larmor frequency, which is proportional to the magnetic field strength at the site of the nucleus. Conventional “T₁” relaxation occurs purely between the three triplet states. The relaxation processes connecting the triplet and singlet manifolds are much weaker, in many circumstances. It is also possible to directly generate hyperpolarized singlet order occurs if the degree of polarization is high enough.

In thermal equilibrium in a magnetic field, the magnetization is around 1 part in 10⁵ (or less), while the singlet polarization is around 1 part in 10¹⁰ (or less).

The methods described above (selective 180 degree pulse followed by transport into low field) convert the thermal equilibrium magnetization into singlet order. So in this case the singlet polarization would become of the order of 1 part in 10⁵.

Generally, hyperpolarization requires a degree of nuclear spin order (which could either be magnetization or singlet order) which is larger than that achievable by thermal polarization in a routinely-accessible magnetic field. For ¹⁵N, this figure is about 1 part in 10⁵. Thus, hyperpolarized ¹⁵N magnetization can be thought of as having a degree of nuclear spin order of greater than one part in 10⁴. Hyperpolarized ¹⁵N singlet order also can be thought of as having a degree of nuclear spin order of greater than one part in 10⁴. In the present invention, the singlet state does not have to be hyperpolarized singlet order, although hyperpolarized singlet order is preferable.

Once the hyperpolarized singlet order is generated, it can be maintained by keeping the sample in a sufficiently weak magnetic field. In the case of [1,2-¹⁵N₂]—N₂O this magnetic field must be less than around 20 milliTesla. Some magnetic shielding may be necessary to ensure this, in the context of an NMR or MRI facility.

The solution of singlet-hyperpolarized [1,2-¹⁵N₂]—N₂O could be administered in the same way as described above for the non-singlet state except that the administering should be performed in a low-magnetic field. This may require the agent to be shielded during the administration step.

When the singlet-hyperpolarized [1,2-¹⁵N₂]—N₂O is moved into the magnetic field (either within the subject, or before administration to the subject), the singlet order is converted naturally into hyperpolarized magnetization, but with opposite signs for the two ¹⁵N sites. A selective 180-degree pulse, of the same type as used for the generation of the singlet state, is applied before a fast NMR imaging sequence is used to construct the NMR image. Many of the imaging sequences used for the non-singlet state may also be used for the singlet state.

The long lifetime of the singlet state will allow a longer time for the hyperpolarized agent to be distributed within the body, compared to the conventionally-hyperpolarized material. This will have advantages for the study of organs which are remote from the site of administration.

To demonstrate the properties of ¹⁵N₂O in solution as an agent, ¹⁵N₂O gas was dissolved in a degassed solution of DMSO-d₆ at a pressure of approximately 3.5 bar. The concentration of the ¹⁵N₂O in solution was approximately 0.3M.

FIG. 3 trace (a) shows the NMR spectra of the above solution taken using a field of B_{high}=7.0463 T. The signal due to both ¹⁵N sites is observed. The splitting of the peaks is due to J-coupling.

FIG. 3 trace (b) is the NMR spectra of the above solution taken after the pulse sequence of FIG. 4 is performed. The pulse sequence of FIG. 4 allows the formation of ¹⁵N₂O with a relatively high degree of singlet order. Due to the long lifetime of the singlet state the solution can be introduced into samples and allowed to fully disperse before the NMR spectra is collected. The long lifetime of the singlet state is achieved by suppressing the interconversion of the singlet state with the three triplet states by maintaining the solution in a low magnetic field, B_{low} which was approximately less than 2 mT in this example.

The singlet state was prepared by allowing the solution to reach equilibrium in a high magnetic field and then applying two strong 90° pulses with a relative phase of 90 at the mean chemical shift frequency of the two ¹⁵N sites. The delay between the pulses τ₁=0.198 ms was chosen so that the transverse magnetization vectors of the two ¹⁵N sites precess through 180° relative to once another. The two pulses act as a selective 180° pulse on one of the ¹⁵N sites. The solution is then moved out of the high magnetic field state to the low magnetic field over a time τₜᵣ=40 s. This slow adiabatic transport out of the high field regime converts the population of each high field state into that of the corresponding low field state leading to a sample with a degree of singlet order. The slow adiabatic transport may be achieved by using a stepper motor.

The sample is then left in the low field region for a variable time τ_{LF}. During the first few minutes, the three triplet populations equilibrate with one another on a time scale set by the relaxation constant T₁. During this time the solution can be dispersed in a sample to be imaged.

The solution is then transported back into the high field region slowly over a time scale to allow adiabatic transport. When the sample is in position in the high field, two strong 90° pulses with a relative phase of 45° are applied, separated by a delay of τ₂=0.099 ms. This acts as a selective 90° pulse on one of the sites which gives rise to an NMR signal. The signal produced is shown in trace (b) of FIG. 3. Using this procedure, the intensity of the single peak was found to be 33% larger than that generated in the conventional spectrum, neglecting relaxation losses.

For trace (b) of FIG. 3, the time τ_{LF} was 300 s, i.e. less than T_{S}. However, for trace (c), the time τ_{LF} was 40 min which is longer than Tₛ and hence the singlet state had relaxed by the time the solution was put back into the high field regime and no NMR signal was measured.

FIG. 4 summarises the field and pulse conditions used above. Here, trace (a) indicates the strength of the magnetic field, trace (b) the RF pulse sequence and trace (c) the timeline.

FIG. 5 is a diagram of the circulatory system of a human.

The long T₁ of ¹⁵N—N₂O and the long T_{S} of the singlet state allows the imaging of both oxygenated and deoxygenated or venous blood. It is well known from studies in, Xenon that the oxygenation level of the blood affects the relaxation time T₁. The lower the blood oxygen level, the shorter T₁ or T_{S} is observed. Also venous blood flows more slowly than arterial blood. Both of these factors work together to make imaging venous blood flow difficult. This makes imaging the flow of venous blood very difficult with conventional MRI agents. However, the long T₁ or T_{S} of ¹⁵N—N₂O provides an agent which can be used to image venous blood flow.

There are known imaging pulse sequences which allow contrast to be observed using T₁. These may be used for both T₁ and T_{S} with ¹⁵N—N₂O for showing contrast between oxygenated and the oxygenated blood.

Further, the relaxation time of the singlet state will be more sensitive to the oxygenation level than the relaxation time of the non-singlet state. Therefore, higher contrast can be obtained using the singlet state relaxation time.

Further, since N₂O is more soluble in fat than in blood, it will partition from the blood into the fat, giving a higher NMR signal from regions in which the blood is in contact with fat. This provides a mechanism for imaging fat deposits in arteries or veins.

The above also has implications of a so-called functional MRI, which is used to measure blood oxygen levels in the brain and to assess brain function.

Blood oxygenation levels and blood flow in the brain are linked to neural activity. It is believed that when nerve cells are active, they consume oxygen carried by haemoglobin and red blood cells from local capillaries. This local response to oxygen utilisation as well as a change in the oxygen level also increases blood flow. Oxyhaemoglobin has a reduced paramagnetism compared to deoxy-haemoglobin. Therefore, the NMR signal of blood is slightly different depending on the level the oxygenation. To date, functional MRI has been performed by exciting an NMR signal from components in the body, usually ¹H.

As described above, the use of ¹⁵N—N₂O allows blood flow to be monitored and also allows the difference between oxygenated and deoxygenated blood to be determined. Therefore, it may also be used for functional MRI since the contrast for oxygenation levels should be larger for hyperpolarized N₂O.

Imaging or investigation of the human or animal body has been discussed above. However, it is possible to use the new agent in all in known medical uses.

It is also possible to use the agent in non-medical uses. For example, it may be used in imaging the flow of oil, image porous rocks to determine oil or other liquid retention or flow patterns within the rocks, or in general applications of the chemical engineering industry. The agent may also be used in the food industry for example for tracking the effectiveness of mixing or other processes used in large scale food production. In these contexts, the use of the singlet state may have particular advantages since the singlet state is less sensitive to the inhomogeneous magnetic fields that are commonly found inside materials such as rocks.

It is also possible to use it for so-called poor man's MRI, where instead of an MRI apparatus, a magnetic field is applied at a point along the pipeline or the like, to excite an MRI signal if a suitable MR agent is present.

Hyperpolarized ¹⁵N—N₂O may also be used as a means for conveying and transporting hyperpolarized nuclear spin order, rather than as an agent itself. For example, any of the hyperpolarized ¹⁵N—N₂O isotopomers could be generated in liquid form using suitable conditions of temperature and pressure (at ambient temperature, N₂O liquefies at around 60 bar). Other molecular species, also containing ¹⁵N or another magnetic isotope, could be dissolved in the liquid hyperpolarized N₂O, and the solution frozen. Known cross-relaxation processes will cause the ¹⁵N hyperpolarization to be transferred from the hyperpolarized ¹⁵N—N₂O to the ¹⁵N-labelled nuclei of the solute. The N₂O could then be evaporated leaving a hyperpolarized solid solute. This could be used in turn for NMR experiments or for hyperpolarized NMR imaging experiments. This procedure may be a more convenient method than direct hyperpolarization of the substance of interest, since it will be easier to rapidly separate the paramagnetic agents from nitrous oxide than from most other chemical substances, since nitrous oxide is a very small linear molecule and hence it is relatively easy to provide a membrane which will allow the passage of nitrous oxide, but block the passage of the paramagnetic species.

1. An agent for magnetic resonance studies, the agent comprising hyperpolarized ¹⁵N—N₂O in solution or liquid ¹⁵N—N₂O.

2. An agent according to claim 1, wherein the hyperpolarized ¹⁵N—N₂O is in the singlet state.

3. An agent according to claim 1, wherein the ¹⁵N—N₂O is [1-¹⁵N]—N₂O, [2-¹⁵N]—N₂O, or [1,2-¹⁵N₂]—N₂O.

4. An agent according to claim 1, wherein the solution comprises at least one selected from water, blood, oxygenated blood, deoxygenated blood, plasma, fat or oil.

5. A method for magnetic resonance studies of a sample using ¹⁵N—N₂O in solution or liquid ¹⁵N—N₂O, comprising:
delivering hyperpolarized ¹⁵N—N₂O in solution or liquid ¹⁵N—N₂O to a predetermined region of the sample;
applying a magnetic field to the sample;
exciting the predetermined region of the sample with an RF excitation pulse suitable for exciting an NMR signal from ¹⁵N—N₂O in the applied magnetic field; and
acquiring magnetic resonance data associated with ¹⁵N—N₂O from the sample.

6. A method according to claim 5, further comprising preparing said hyperpolarized ¹⁵N—N₂O.

7. A method according to claim 6, wherein said hyperpolarized ¹⁵N—N₂O is prepared via dynamic nuclear polarisation.

8. A method according to claim 6, further comprising transferring said hyperpolarized ¹⁵N—N₂O to said sample under a magnetic field which is larger than the magnetic fluctuations in the environment of the sample.

9. A method according to claim 5, further comprising preparing singlet state ¹⁵N—N₂O.

10. A method according to claim 9, further comprising transferring said singlet state ¹⁵N—N₂O to said sample under shielding such that the magnetic field around said ¹⁵N—N₂O is reduced to at most 50 mT.

11. A method according to claim 9, further comprising applying a selective 180 degree RF pulse to said ¹⁵N—N₂O and after said ¹⁵N—N₂O has come under the magnetic field applied to the sample, said selective 180 degree RF pulse being at a frequency resonant with one of the two ¹⁵N sites.

12. A method according to claim 5, wherein the hyperpolarized ¹⁵N—N₂O is provided in a solution of blood, water, blood, oxygenated blood, deoxygenated blood or plasma.

13. A method according to claim 5, further comprising measuring the chemical shift of the acquired magnetic resonance data.

14. A method according to claim 5, where the is [1,2-¹⁵N₂]—N₂O, the method further comprising, adapting the RF excitation pulse to combine or separate the data from the two ¹⁵N sites.

15. A method according to claim 5, wherein the agent is ¹⁵N—N₂O in solution and the sample is a human or animal and the method comprising imaging the flow of blood containing the agent.

16. A method according to claim 15, further comprising imaging the flow of venous blood.

17. A method according to claim 5, wherein the agent is ¹⁵N—N₂O in solution and the method further comprises imaging the brain.

18. Use of hyperpolarized ¹⁵N—N₂O in solution or liquid ¹⁵N—N₂O as an agent in the methods of claim 5.

19. Use of hyperpolarized ¹⁵N—N₂O in solution or liquid ¹⁵N—N₂O as an agent in magnetic resonance studies.

20. An MRI apparatus comprising:
means to apply a magnetic field to a sample;
means to apply RF radiation to the sample; and
means to detect an NMR signal arising from the sample due to the application of the magnetic field and RF radiation, wherein the means to apply a magnetic field and the means to apply RF radiation are configured to excite an NMR signal from ¹⁵N—N₂O and the means to detect an NMR signal are configured to detect an NMR signal from ¹⁵N—N₂O.

21. An MRI apparatus according to claim 20, further configured to apply a 180 degree RF pulse at a frequency resonant with one of the two ¹⁵N sites of ¹⁵N—N₂O.

22. An MRI apparatus according to claim 20, configured to collect data indicating the flow of a liquid.

23. An MRI apparatus according to claim 20, further configured to measure the chemical shift in the detected NMR signal.

24. An MRI apparatus according to claim 20, further configured to apply a sequence of pulses of RF radiation suitable for distinguishing between the signals generated by each of the ¹⁵N sites in [1,2-¹⁵N₂]—N₂O.

25. A method of preparing an MRI agent, the method comprising:
hyperpolarizing ¹⁵N—N₂O, and dissolving said ¹⁵N—N₂O in solution.

26. A method according to claim 25, further comprising preparing singlet state ¹⁵N—N₂O.

27. A method according to claim 25, wherein the ¹⁵N—N₂O is hyperpolarized using a paramagnetic dopant and ¹⁵N—N₂O is removed from said dopant using a membrane separation method or chromatography.

28. A method of hyperpolarizing a substance, the method comprising:
liquefying hyperpolarized ¹⁵N—N₂O,
dissolving the substance to be hyperpolarized in said liquefied ¹⁵N—N₂O; and
removing said ¹⁵N—N₂O.

29. A method according to claim 28, wherein said substance comprises ¹⁵N or ¹³C nuclei.
